(19) United States
(12) Patent Application Publication
(10) Pub. No.: US 2018/0056757 A1
(43) Pub. Date: Mar. 1, 2018

(54) USB PLUG IN VEHICLE AIR FRESHENER

(71) Applicant: Jonathan Murphy, North Hero, VT (US)

(72) Inventor: Jonathan Murphy, North Hero, VT (US)

(21) Appl. No.: 15/249,761

(22) Filed: Aug. 29, 2016

(51) Int. Cl. B60H 3/00; A61L 9/03

(52) U.S. Cl. CPC B60H 3/0035; A61L 9/03; B60H 2003/005; A61L 2209/11; A61L 2209/13

(57) ABSTRACT

A USB plug in vehicle air freshener for dispensing a fragrance in a host vehicle. The vehicle air freshener has an elongated freshener body that includes on its exterior a USB jack for plugging into a USB plug on the host vehicle at one end and a vent as the other end, as well as an exterior manual activating switch. On the interior, the freshener body includes a fragrance that hold a fragrant material to be dispensed, a heating element for vaporizing the fragrant material so that it may exit through the vent, and an internal control system that allows the dispensing of fragrance to be controlled wirelessly by a separate device, such as a computer system on the host vehicle.

### BACKGROUND OF THE INVENTION

### Field of the Invention

This invention relates generally to fragrant emitting device and, more particularly, to a vehicle air freshener operative to plug into a USB port.

### Description of the Prior Art

The use of conventional air fresheners that mask ambient odors by emitting a fragrance is well known. For example, the use incense and scented oils to dispense a fragrance through combustion is believed to date back thousands of year. In modern times, various sprays or electrical dispensing devices are often used to dispense a fragrance in an area.

A problem which still exists, however, is that many existing air fresheners which are designed for a vehicle do not fully integrate with the vehicle's existing control systems. Thus, there remains a need for a USB plug in vehicle air freshener operative to disperse a fragrant material into a vehicle using electricity supplied by the vehicle. It would be helpful if such a USB plug in vehicle air freshener included a built in fragrance reservoir to maximize the consistent dispensing of fragrance. It would be additionally desirable for such a USB plug in vehicle air freshener to be selectively activated from either a manual switch or a signal from the vehicle.

The Applicant's invention described herein provides for a USB plug in vehicle air freshener adapted to allow a user to dispense a fragrance in a vehicle through electrically driven mechanisms. The primary components in Applicant's USB plug in vehicle air freshener are a freshener body, an internal control system, a reservoir, a vent, and a manual switch. When in operation, the USB plug in vehicle air freshener provides better control over the dispensing of fragrance in a vehicle. As a result, many of the limitations imposed by prior art structures are removed.

### SUMMARY OF THE INVENTION

A USB plug in vehicle air freshener for dispensing a fragrance in a host vehicle. The vehicle air freshener comprises an elongated freshener body that includes on its exterior a USB jack for plugging into a USB plug on the host vehicle at one end and a vent as the other end, as well as an exterior manual activating switch. On the interior, the freshener body includes a fragrance that hold a fragrant material to be dispensed, a heating element for vaporizing the fragrant material so that it may exit through the vent, and an internal control system that allows the dispensing of fragrance to be controlled wirelessly by a separate device, such as a computer system on the host vehicle.

It is an object of this invention to provide a USB plug in vehicle air freshener operative to disperse a fragrant material into a vehicle using electricity supplied by the vehicle.

It is another object of this invention to provide a USB plug in vehicle air freshener that includes a built in fragrance reservoir to maximize the consistent dispensing of fragrance.

It is yet another object of this invention to provide a USB plug in vehicle air freshener that is selectively activated from either a manual switch or a signal from the vehicle.

These and other objects will be apparent to one of skill in the art.

### BRIEF DESCRIPTION OF THE DRAWINGS

FIG. 1 is a top plan view of a USB plug in vehicle air freshener built in accordance with the present invention.

### DETAILED DESCRIPTION OF THE INVENTION

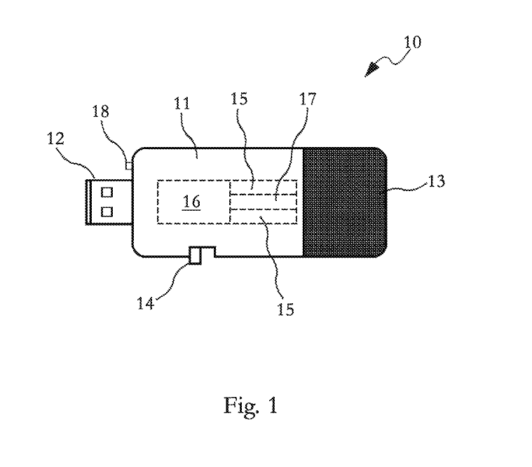

Referring now to the drawings and in particular FIG. 1, a USB plug in vehicle air freshener 10 is shown having an elongated freshener body 11 that includes a USB jack 12 at one end and a vent 13 as the other end. In one embodiment, the USB jack 12 defines a conventional Universal Serial Bus plug that is operative to receive electrical power when plugged into a conventional Universal Serial Bus port and the vent 13 defines a mesh surface which allows gas to freely pass between the interior of the body 11 to the exterior of the body. The body 11 additionally includes a manual activating interface 14 which provides manual control of an internal electrical switch of an internal control system 16 which selectively makes a circuit between the USB jack 12 and the internal control system of the vehicle air freshener 10, so as to supply electricity thereto (when available at the USB jack 12), and breaks the circuit between the USB jack 12 and the internal control system of the vehicle air freshener 10, so as to interrupt the supply of electricity thereto.

Disposed inside the body 11 is a fragrance reservoir 15 that has an open end and holds a fragrant material in gel form, the internal control system 16, and a heating element 17. In one embodiment, the internal control system 16 defines a plurality of electrically interconnected internal electrical components, including the electrical switch, a controller, and a wireless networking interface. The internal control system 16 is electrically connected to the heating element 17 so as to allow the controller to selectively cause electricity supplied to the internal control system 16 to be directed to the heating element 17 to cause the heating element 17 to heat up. As the heating element 17 is positioned adjacent to the fragrance reservoir 15, the heating of the heating element 17 causes the fragrant material in the reservoir to slowly vaporize and exit through the vent 13.

In one embodiment, the networking interface defines a Bluetooth interface and allows the vehicle air freshener 10 to pair (using a pair initiating button 18) with a Bluetooth interface of a host vehicle so as to form a bond therewith that allows the host vehicle and the controller of the vehicle air freshener 10 to wirelessly communicate electrical signals. In this regard, once the vehicle air freshener 10 is bonded with a target vehicle, the controller of the vehicle air freshener 10 may selectively cease directing electricity to the heating element 17 in response to a signal from the host vehicle (and subsequently recommence directly electricity upon receipt of another signal from the host vehicle).

It is contemplated that the vehicle air freshener 10 must be plugged into a USB port and the manual activating interface 14 must be set to supply electricity to the internal control system 16 for the vehicle air freshener 10 to receive and act on signals from the host vehicle. As such, in one embodiment, the default operation of the vehicle air freshener 10 causes electricity supplied to the internal control system to be automatically directed to the heating element and continually direct to the heating element in the absence of receiving an electrical signal from the from a bonded device over a wireless network connection.

It is understood that substantially any fragrance may be used in the fragrance reservoir 15.

The instant invention has been shown and described herein in what is considered to be the most practical and preferred embodiment. It is recognized, however, that departures may be made therefrom within the scope of the invention and that obvious modifications will occur to a person skilled in the art.

What is claimed is:

1. A vehicle air freshener, comprising:
a freshener body having a mechanical connection interface and a vent, wherein said mechanical connection interface is configured to mechanically engage a power source so as to receive electricity;
a fragrance reservoir integral with said freshener body, wherein said fragrance reservoir is adapted to hold a fragrant material in a non-gas form and dispense said fragrant material through the vent if vaporized;
a heating element integral with said freshener body, wherein said heating element is operative to generate heat when supplied with electricity and said heating element is positioned such that heat therefrom causes the fragrant material in the fragrance reservoir to vaporize; and
an internal control system defining at least an electric switch, wherein said internal control system is configured to selectively cause electricity received by the mechanical connection interface to be directed to the heating element by way of the internal control system.

2. The vehicle air freshener of claim 1, additionally comprising a manual activating interface positioned on said body, wherein said manual activating interface is operative to control the state of the electric switch.

3. The vehicle air freshener of claim 1, wherein said internal control system additionally includes a controller and a networking interface and said internal control system is additionally configured to cease directing electricity received by the mechanical connection interface to the heating element in response to an electrical signal received over the networking interface.

4. The vehicle air freshener of claim 3, wherein said networking interface defines a Bluetooth antenna.

5. The vehicle air freshener of claim 1, wherein said mechanical connection interface is Universal Serial Bus plug.

6. A vehicle air freshener, comprising:
a freshener body having a Universal Serial Bus plug and a vent, wherein said Universal Serial Bus plug is configured to mechanically engage a power source so as to receive electricity;
a fragrance reservoir integral with said freshener body, wherein said fragrance reservoir is adapted to hold a fragrant material in a non-gas form and dispense said fragrant material through the vent if vaporized;
a heating element integral with said freshener body, wherein said heating element is operative to generate heat when supplied with electricity and said heating element is positioned such that heat therefrom causes the fragrant material in the fragrance reservoir to vaporize;
a manual activating interface positioned on said body, wherein said manual activating interface is operative to control the state of the electric switch; and
an internal control system defining at least an electric switch, wherein said internal control system is configured to selectively cause electricity received by the Universal Serial Bus plug to be directed to the heating element by way of the internal control system.

7. The vehicle air freshener of claim 6, wherein said internal control system additionally includes a controller and a networking interface and said internal control system is additionally configured to cease directing electricity received by the mechanical connection interface to the heating element in response to an electrical signal received over the networking interface.

8. The vehicle air freshener of claim 7, wherein said networking interface defines a Bluetooth antenna.
